(19) United States
(12) Patent Application Publication
(10) Pub. No.: US 2017/0321228 A1
(43) Pub. Date: Nov. 9, 2017

(54) Method for Enhancing Microbial Methane Produciton

(71) Applicants: William H Orem, Oak Hill, VA (US); Elizabeth Jones, Manassas, VA (US); Arthur Clark, Pine, CO (US); Elliott Barnhart, Clancy, MT (US); Leslie Ruppert, Arlington, VA (US); Denise Akob, Reston (UD)

(72) Inventors: William H Orem, Oak Hill, VA (US); Elizabeth Jones, Manassas, VA (US); Arthur Clark, Pine, CO (US); Elliott Barnhart, Clancy, MT (US); Leslie Ruppert, Arlington, VA (US); Denise Akob, Reston (UD)

(73) Assignee: United States Department of the Interior, Washington DC, DC (US)

(21) Appl. No.: 15/604,986

(22) Filed: May 25, 2017

Related U.S. Application Data
(60) Provisional application No. 62/333,620, filed on May 9, 2016.

(51) Int. Cl. C12P 5/02; E21B 47/00; E21B 43/16; E21B 49/08; E21B 43/00

(52) U.S. Cl. CPC C12P 5/023; E21B 49/08; E21B 43/006; E21B 43/16; E21B 47/00

(57) ABSTRACT

A process for revitalization of the coal deposit for enhanced microbial gas production by exposing coal deposit to air oxidation. Water is removed from coal beds and stored. The coal is exposed to hydrogen peroxide for providing oxygen-rich organic molecules that are more readily biodegraded. Oxidation and drying out of coal produces additional cracks within the coal matrix to allow microbes greater access to the overall coal matrix. The coal is reinjected with emulsified oil and inorganic nutrients by transporting produced water from the storage sites to the reinjection wells. Produced water is mixed with oil and inorganic nutrients in a holding tank. The reinjection water containing nutrients and oil is then injected into the coal under pressure. The mixture presence of the oil speeds up cell division of bacteria and methanogens in the coal and create microbial biomass sufficient to accelerate biomethanogenesis of the coal.

### STATEMENT REGARDING FEDERALLY SPONSORED RESEARCH OR DEVELOPMENT

The invention described herein was made by an employee of the United States Government and may be manufactured and used the Government of the United States of America for governmental purposes without the payment of any royalties thereon or therefore.

### FIELD OF INVENTION

This invention relates to the field of methane production and more specifically to producing methane in situ in a coal or shale bed using microbes.

### BACKGROUND OF THE INVENTION

Unconventional sources of methane gas have become an important component of the energy industry. As more conventional sources of energy become increasingly scarce, these unconventional sources such as coal bed methane gas, shale bed methane gas, and microbial methane gas from coal and shale will become increasingly important.

Methane gas is formed both as a thermogenic product of coal formation as well as by microbiological activity. Microbial gas in coal beds and shale has been exploited in places like the Powder River Basin, Wyo. This gas accumulated over long time periods as naturally-occurring microbes it the coal or shale biodegraded geopolymers in the fossil energy deposits to methane gas.

While coal represents the most abundant energy resource in the USA, it suffers from a number of disadvantages as an energy resource, including: producing many different contaminants when burned, landscape alteration from mining activities, and lower BTU yield per unit burned compared to other fossil energy resources such as oil and gas. The potential for generating new biogenic methane from coal deposits may provide a new use for coal. Methane burns cleanly and can be produced more economically and with lower environmental impact than coal. Also, coal deposits that are inaccessible or too expensive to mine under current conditions might be able to produce microbial methane.

A preliminary biodegradation pathway for the biodegradation of geopolymers in coal to methane gas has multiple steps, with different microorganisms and organic intermediates involved in each step. The initial step involves release of monomeric, long-chain organic intermediates from the coal geopolymers. These compounds include long-chain fatty acids (LCFA), aromatic substances, and long-chain alkanes. The first step in the process is likely to be the rate determining step because of the difficulty in biodegrading the relatively refractory coal geopolymers. A second step involves biodegradation of the LCFA to smaller fatty acids of mid chain length, and a third step involves further biodegradation of these mid chain length fatty acids to simple molecules like acetate or hydrogen gas able to be used by methanogens. The final step in the process is the production of methane by the methanogens.

The process of biodegradation of coal geopolymers in the field is slow, and limited by a number of factors, including: low levels of inorganic nutrients, low microbial biomass, high salinity in some cases, and the condensed nature of coal that limits microbial biodegradation to fractures in the coal. Laboratory studies have also suggested that although LCFA are necessary intermediates in the biodegradation pathway, buildup of these substances to levels that are too high may inhibit methanogenesis.

There is an unmet need in the art for methods which efficiently produce methane using methanogens.

There is a further unmet need in the art for methods which can stimulate methane production in environments where naturally-occurring methane has already been removed.

### BRIEF DESCRIPTION OF SEVERAL VIEWS OF THE DRAWING(S)

FIGS. 1a and 1b are a flowchart illustrating an exemplary embodiment of a method for enhancing microbial methane production.

### TERMS OF ART

As used herein, the term “methane” means an alkane with a chemical formula of CH₄.

### DETAILED DESCRIPTION OF THE INVENTION

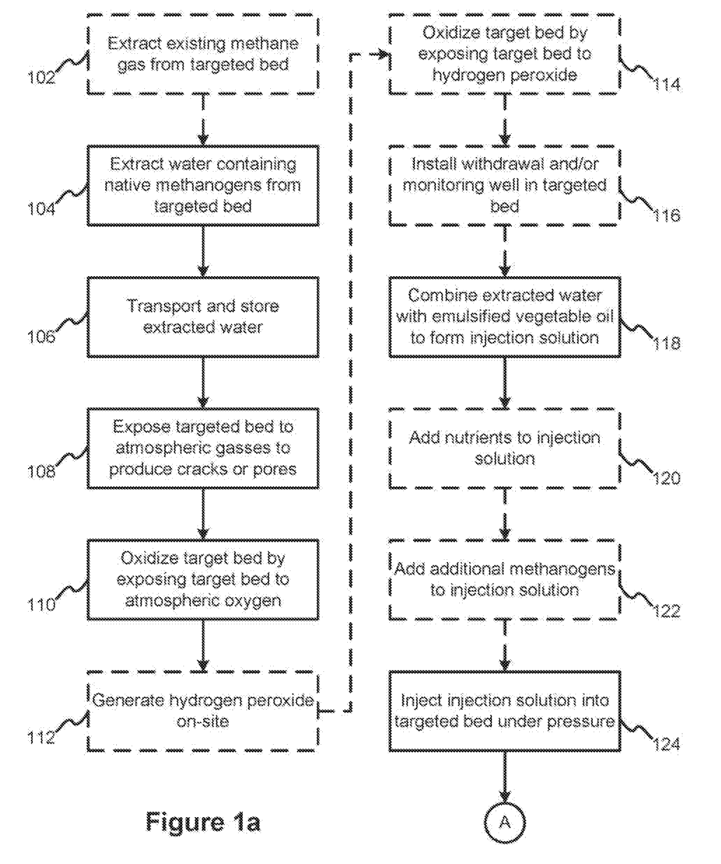

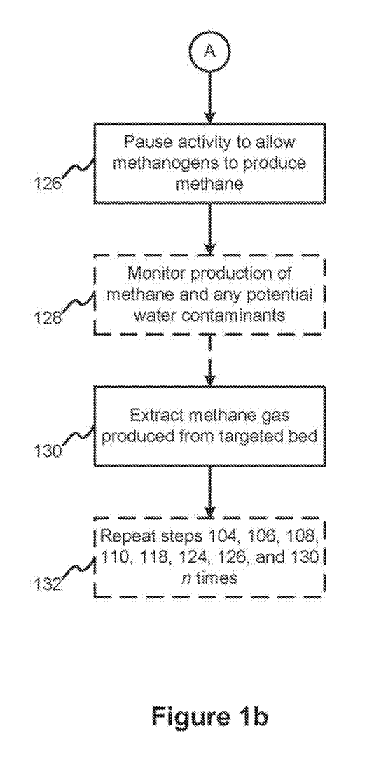

FIGS. 1a and 1b are a flowchart illustrating an exemplary embodiment of method 100 for enhancing microbial methane production.

In optional step 102, method 100 extracts existing methane gas from a targeted bed. The targeted bed may be a coal or shale bed.

In step 104, method 100 extracts water containing native methanogens from the targeted bed. Steps 102 and 104 may be performed simultaneously.

In step 106, method 100 transports and stores the extracted water in a storage unit such as a holding tank or pond. Notably, this step does not kill or remove native methanogens such as methanogenic bacteria and archaea from the extracted water.

In step 108, method 100 exposes the targeted bed to atmospheric gasses produce cracks or pores within the coal or shale matrix. This increased porosity allow methanogens greater access to and penetration of the coal or shale matrix.

In step 110, method 100 oxidizes the target bed by exposing the target bed to atmospheric oxygen. Oxidation usually requires approximately six months for oxygen to react with the geopolymers in the targeted bed and produce oxygen-rich organic molecules that are more readily biodegraded by methanogens.

In optional step 112, method 100 generates hydrogen peroxide (H₂O₂) on-site using portable reaction vessels. In the exemplary embodiment, step 112 reacts hydrogen (H₂) gas with anthraquinone using a palladium catalyst to produce anthraquinol. The anthraquinol solution is filtered to remove the catalyst particles, and then reacted with air to produce a solution of approximately 40% hydrogen peroxide.

In optional step 114, method 100 oxidizes the target bed by exposing the target bed to hydrogen peroxide. In the exemplary embodiment the hydrogen peroxide is a solution of approximately 5% and includes extracted water.

In optional step 116, method 100 installs at least one withdrawal and/or monitoring well in or proximal to the targeted bed. Monitoring wells are located downslope of the targeted bed to allow users to monitor water quality within the targeted bed aquifer, and in the geological strata above and below the targeted bed undergoing method 100 to monitor methane production. This step may not be necessary in targeted beds with existing withdrawal and/or monitoring wells from previous resource extractions.

In step 118, method 100 combines the extracted water with emulsified vegetable oil (EVO) to form an injection solution. In the exemplary embodiment, the EVO is a solution of approximately 5% with the extracted water. The EVO may include, but not limited to, rapeseed oil, canola oil, soybean oil, corn oil, sunflower oil, safflower oil, peanut oil, olive oil, nut oil, or any other vegetable oil known in the art.

In optional step 120, method 100 adds at least one nutrient to the injection solution. These nutrients may include, but are not limited to, sodium bicarbonate (NaHCO₃), ammonium chloride (NH₄Cl), monosodium phosphate (NaH₂PO₄), potassium chloride (KCl), vitamins, and/or minerals. In the exemplary embodiment, the nutrients added to the injection solution include 2.5 g/L of sodium bicarbonate, 0.5 g/L of ammonium chloride, 0.5 g/L of monosodium phosphate, 0.1 g/L of potassium chloride, and trace minerals and vitamins at ppb levels.

In optional step 122, method 100 adds additional at least one additional methanogen population to the injection solution. Such microbes may include bacteria and archaca microbes.

In step 124, method 100 injects the injection solution into the targeted bed under pressure.

In step 126, method 100 pauses activity for an interval of time to allow the methanogens in the injection solution to produce nethane. This interval may be predetermined or based on monitoring conducted in step 128.

In optional step 128, method 100 monitors production of methane and any potential water contaminants. Water contaminant parameters of interest will include nutrients, metals, and potentially toxic organic substances (e.g. polycyclic aromatic hydrocarbons derived from coal), and methanogens. Steps 126 and 128 may be performed simultaneously,

In step 130, method 100 extracts methane gas produced by method 100 from the targeted bed.

In optional step 132, method 100 repeats at least steps 104, 106, 108, 110, 118, 124, 126, and 130 n number of times.

Method 100 has potential application to coal and shale that contains organic matter. It can be used for coal beds in which coal is unlikely to be mined for a number of reasons. Method 100 can be used for a number of cycles of gas generation, though the number of practicable cycles may vary from one coal bed to another.

It will be understood that many additional changes in the details, materials, procedures and arrangement of parts, which have been herein described and illustrated to explain the nature of the invention, may be made by those skilled in the art within the principle and scope of the invention as expressed in the appended claims.

It should be further understood that the drawings are not necessarily to scale; instead, emphasis has been placed upon illustrating the principles of the invention. Moreover, the term “approximately” as used herein may be applied to modify any quantitative representation that could permissibly vary without resulting in change the basic function to which it is related.

What is claimed is:

1. A method for enhancing microbial methane production, comprising the steps of:
(i) extracting water containing native methane from a targeted bed;
(ii) transporting and storing said water and said native methanogens;
(iii) exposing said targeted bed to atmospheric gasses to produce cracks or pores in a matrix in said targeted bed;
(iv) exposing said targeted bed to atmospheric oxygen to oxidize said targeted bed:
(v) combining said and said native methanogens with emulsified vegetable oil (EVO) to form an injection solution;
(vi) injecting said injection solution into said targeted bed under pressure;
(vii) pausing for an interval of time to allow said native methanogens to produce methane gas; and
(viii) extracting said methane gas from said targeted bed.

2. The method of claim 1, further comprising the step of repeating step (i)-(viii) n times.

3. The method of claim 1, further comprising the step of extracting existing methane gas from said targeted bed before step (i).

4. The method of claim 1, further comprising the step of exposing said targeted bed to hydrogen peroxide to oxidize said targeted bed before step (v).

5. The method of claim 4, further comprising the step of generating said hydrogen peroxide on site using portable reaction vessels.

6. The method of claim 4, wherein said hydrogen peroxide is a 5% solution.

7. The method of claim 1, further comprising the step of installing at least one withdrawal well in said targeted bed before step (vi).

8. The method of claim 1, further comprising the step of installing at least one monitoring well proximal to said targeted bed before step (vi).

9. The method of claim 8, wherein said at least one monitoring well is installed downslope of said targeted bed.

10. The method of claim 1, further comprising the step of adding at least one nutrient to said injection solution before step (vi).

11. The method of claim 10, wherein said at least one nutrient is selected from the group consisting of: sodium bicarbonate (NaHCO₃), ammonium chloride (NH₄Cl), monosodium phosphate (NaH₂PO₄), potassium chloride (KCl), vitamins, and minerals.

12. The method of claim 1, further comprising the step of adding at least one additional methanogen population to said injection solution before step (vi).

13. The method of claim 1, further comprising the step of monitoring production of methane gas after step (vi).

14. The method of claim 13, wherein said interval of time is determined by a level of methane gas said targeted bed.

15. The method of claim 1, further comprising the step of monitoring water contaminants after step (vi).

16. The method of claim 15, wherein said water contaminants are selected from the group consisting of: nutrients, metals, potentially toxic organic substances, methanogens, and non-methanogenic microbes.

17. The method of claim 1, wherein said targeted bed is selected from the group consisting of: a coal bed and a shale bed.

18. The method of claim 1, wherein said EVO is a 5% solution.

19. The method of claim 1, wherein said EVO is selected from the group consisting of: rapeseed oil, canola oil, soybean oil, corn oil, sunflower oil, safflower oil, peanut oil, olive oil or nut oil.

20. The method of claim 1, wherein said interval of time is predetermined.
